(19) United States
(12) Patent Application Publication
(10) Pub. No.: US 2026/0305997 A1
(43) Pub. Date: Oct. 8, 2026

(54) AI-POWERED COGNITIVE HEALTH ASSESSMENT AND MANAGEMENT SYSTEM AND METHOD

(71) Applicant: SCIENZA HEALTH, INC., NEWPORT BEACH, CA (US)

(72) Inventor: DAVID A KAISER, NEWPORT BEACH, CA (US)

(73) Assignee: SCIENZA HEALTH, INC., NEWPORT BEACH, CA (US)

(21) Appl. No.: 19/296,870

(22) Filed: Aug. 11, 2025

Related U.S. Application Data
(60) Provisional application No. 63/681,276, filed on Aug. 9, 2024.

(51) Int. Cl. A61B 5/16; A61B 5/00; A61B 5/0205; A61B 5/024; A61B 5/11; G06F 21/62; G16H 10/20; G16H 10/60; G16H 50/30

(52) U.S. Cl. CPC A61B 5/165; A61B 5/0205; A61B 5/1118; A61B 5/4803; A61B 5/4812; A61B 5/681; G06F 21/6245; G16H 10/20; A61B 5/02405; G16H 10/60; G16H 50/30

(57) ABSTRACT

Compute based systems and method for AI-powered cognitive health assessment and management platform using multi-modal data integration, digital twin framework, and real-time streaming analytics. The present invention provides a comprehensive AI-powered cognitive health assessment platform that overcomes limitations of prior art through several key innovations. A computer-implemented system for cognitive health assessment including a multi-modal data integration module configured to receive and process cognitive health related data in substantially real time. The system also includes a digital twin framework maintaining a comprehensive patient cognitive health model and accepts data from said multi-modal data integration module. A real-time streaming analytics engine is included that accepts data from said digital twin framework and a clinical trial matching system is included that accepts data from the digital twin framework, wherein said system generates cognitive assessments in substantially real-time.

This application claims the benefit of U.S. Provisional Pat. Appl. Ser. No. 63/681,276 filed on Aug. 9, 2024.

### BACKGROUND OF THE INVENTION

### Field of the Invention

This invention relates to artificial intelligence (AI) based systems for healthcare, and in particular to cognitive health assessment platforms that integrate multiple data modalities including but not limited to, voice biomarkers, wearable device biometrics, and electronic health records for real-time cognitive status evaluation and clinical decision support.

### Description of Related Art

Current cognitive assessment systems and methods suffer from several limitations. For example, existing systems can rely on fragmented data sources that typically rely on single data modalities (e.g., only voice analysis or only clinical assessments). These conventional systems and methods can suffer from delayed processing because assessments require manual scoring and interpretation. This can not only be frustrating for the patient but can lead to delays in diagnosis and treatment decisions. Current approaches can also rely on periodic clinical visits rather than continuous monitoring of cognitive health indicators. These visits may only give a small window into the day-to-day condition of the patient, and what may be the best diagnosis or treatment.

Wearable electronic devices have become developed and have become more popular, and the devices can collect extensive biometric data of the wearer. Existing healthcare systems lack sophisticated algorithms to correlate this data with cognitive health indicators and utilize it with other indicators to determine the best diagnosis and treatment.

There is a need for participants in clinical trials and often these participants must meet certain qualifications and characteristics. There are currently insufficient systems for matching the qualitied with particular clinical trials. Patients with cognitive decline often miss opportunities for clinical trial participation due to inadequate matching algorithms and geographic constraints.

U.S. Pat. No. 10,354,753 to Lawrence et al. (Medical Failure Pattern Search Engine) discloses a patient safety search engine and alarm processor is programmed to repetitively search the electronic medical records of all patients in a hospital system to automatically provide early detection of patients with evolving pathophysiologic cascades, and in particular the cascades of evolving death, such as cascades of septic shock.

U.S. Pat. No. 9,848,776 to Proud (Methods Using Activity Manager for Monitoring User Activity) discloses a method using telemetry data based on user habit information or user monitoring where user information is acquired from one or more sensors of a monitoring device.

U.S. Patent Appl. Ser. No. 2007/005555A1 to Clair et al. (System and Method for Health Care Data Integration and Management) discloses a method creates an electronic health record and analyzes that record to present a summary report. The report may optionally include treatment opportunities, strategies, and plans for the physician and patient. Data processing steps used to create and analyze the record and deliver the summary data may include aggregation, integration, internal validation, clinical validation, inspection, prediction, and communication.

### SUMMARY OF THE INVENTION

The present invention provides a comprehensive cognitive health assessment platform that overcomes the limitations of prior art. Systems and methods according to the present inventions can operate in many different ways with some embodiments being machine language or AI based or powered. The present invention is directed to new and novel cognitive health assessment systems and methods that utilize a combination of multi-modal real-time processing and digital twin architecture, systems and methods. The present invention also provides for new and novel intelligent clinical trial matching with qualified patients.

Some embodiments of the present invention can comprise and AI-powered cognitive health assessment and management platform using multi-modal data integration, digital twin framework, and real-time streaming analytics. The present invention provides a comprehensive AI-powered cognitive health assessment platform that overcomes limitations of prior art through several key innovations.

One embodiment of the present invention comprises a computer-implemented system for cognitive health assessment including a multi-modal data integration module configured to receive and process cognitive health related data in substantially real time. The system also includes a digital twin framework maintaining a comprehensive patient cognitive health model and accepts data from said multi-modal data integration module. A real-time streaming analytics engine is included that accepts data from said digital twin framework and a clinical trial matching system is included that accepts data from the digital twin framework, wherein said system generates cognitive assessments in substantially real-time.

One embodiment of a computer-implemented cognitive health assessment platform includes a multi-modal engine utilizes artificial intelligence and accepts real-time patient cognitive health related data. A digital twin framework is included that accepts data from the multi-modal engine, and a real-time streaming analytics engine is included that accepts data from said digital twin framework. An avatar interface integration subsystem is included that can real-time conversational AI and can real-time patient cognitive health related data to said multi-modal engine, wherein the platform generates and reports substantially real time cognitive assessments.

One method for generating cognitive health assessment according to the present invention comprises generating cognitive real-time health related data from patient wearable sensors and from avatar interface integration subsystem to generate substantially real-time multi-mode data. Utilizing the multi-mode data to generate a cognitive digital twin of the patient. Utilizing the multi-mode data for clinical trial matching. Generating a substantially real-time cognitive index based on the multi-mode data, with the cognitive index used in generating substantially real-time cognitive assessments.

The systems according to the present invention can comprise many different features, components and subsystems as described below. These and other aspects and advantages of the invention will become apparent from the following detailed description and the accompanying drawings which illustrate by way of example in the features of the invention.

### BRIEF DESCRIPTION OF THE DRAWINGS

FIG. 1A is the first portion of a block and information flow diagram for one embodiment of a cognitive health assessment and management system according to the present invention;

FIG. 1B is the second portion of block and information flow diagram for one embodiment of a cognitive health assessment and management system according to the present invention;

FIG. 2A is the first portion of a block and information flow diagram for the Multi-Modal Cognitive Index Computation subsystem in FIG. 1;

FIG. 2B is the second portion of a block and information flow diagram for the Multi-Modal Cognitive Index Computation subsystem in FIG. 1

FIG. 3A is the first portion of a block and information flow diagram for the digital twin framework in FIG. 1; and

FIG. 3B is the second portion of a block and information flow diagram for the digital twin framework in FIG. 1.

### DETAILED DESCRIPTION OF THE INVENTION

This invention relates to cognitive health assessment platforms, systems and methods that can integrate multiple data modalities including, but not limited to, voice biomarkers, wearable device biometrics, and electronic health records (and other data modalities) to provide a real-time cognitive status evaluation and clinical decision support. The platforms, systems and methods according to the present invention can be implemented using software languages and systems, with the software being stored in known memory storage devices and executed using known processors. In some embodiments the software can reside in the cloud, or on a server, personal computer or smart phone. Some embodiments of the present invention comprise a system component or subsystem interaction flow where the components systems the systems interact through a sophisticated event-driven architecture. Embodiments can comprise different components and subsystems making up the overall systems and some of these can include the following:
A Multi-Modal Cognitive Index Computation subsystem where different functions are performed such as where wearable sensors and voice interactions are monitored and used to generate outputs such as real time and current cognitive index;
A Real-Time Streaming Analytics subsystem where different functions are performed such as a streaming analytics process multi-modal data with sub-second latency;
A Healthcare Digital Twin subsystem where different functions are performed such as maintaining a digital twin comprehensive patient cognitive profiles;
A Clinical Trial Matching System subsystem where different functions are performed such as clinical integration through clinical trial matching; and
An Avatar Interface Integration subsystem where different functions are performed such as real-time conversational AI to determine the condition of a patent such as by determining emotion and other patient conditions during cognitive assessments.

The systems can have many other subsystems such as a Security Overlay subsystem to protect the patient's medical information used and generated with the systems according to the present invention, such as HIPAA-compliant security ensures privacy and regulatory compliance.

The systems according to the present invention provides a number of advantages over the prior art including, improved cognitive assessment accuracy. In some embodiments this accuracy can be up to 96.8% cognitive assessment accuracy compared to 85-90% or less for single-modality systems. The systems also provide for improved real-time processing and in some embodiments, average latency may be on the order of approximately 322 ms verses 5-10 seconds for traditional systems Some embodiments also provide comprehensive monitoring with 24/7 continuous assessment verses conventional periodic clinical visits. The systems also provide a scalable architecture that is capable of supporting more that 1 million patients and active digital twins verses thousands for existing systems. The systems according to the present invention allows for efficient clinical Integration by providing FHIR-compliant EHR integration with real-time provider notifications.

The invention comprises several interconnected subsystems working in concert to provide timely and comprehensive cognitive health assessment. The systems enable substantially seamless integration while maintaining modularity, scalability, and clinical effectiveness. These subsystems are discussed in more detail below.

### Multi-Modal Cognitive Index Computation

The multi-modal cognitive index computation subsystem provides a novel algorithm that integrates different patient relevant information such as voice biomarkers, wearable watch biometric data, electronic health records, and imaging data to compute a unified cognitive health index with high accuracy, with some embodiments providing an accuracy of up to or greater than 95% accuracy. It is understood that different embodiments of the present invention can provide accuracies that are higher or lower than 95%.

This subsystem provides a significant improvement in cognitive assessment by integrating disparate data sources into a unified, clinically meaningful metric. This innovation addresses the fundamental limitation of prior systems that can rely on single or only a couple data modalities. These data sources can come from many different locations and devices with the following being only some of the many sources that can be utilized in different embodiments according to the present invention.

A voice biomarker integration generator can be used that can monitor and extract voice acoustics characteristics from the patents. Many different generators can be such as commercially available Canary Speech API, which can extract up to approximately 500 or more acoustic features including prosody, semantic fluency, and phonetic analysis. These Features are processed through Real-time audio capture, noise reduction spectral analysis, mel-frequency cepstral coefficients (MFCCs), and linguistic pattern recognition. Linguistic analysis provides natural language processing for semantic fluency, word-finding difficulty, and syntactic complexity. Cognitive correlation utilizes algorithms correlating voice features with cognitive domains (memory, attention, executive function) and supports 12+ languages with cultural adaptation. HIPAA-compliant audio processing can also be provided with end-to-end encryption.

One or more wearable or on-patient sensors can be used us to monitor, record and/or report the patient's data related to the patient's health condition in real-time. Many different wearable sensors have been developed such as watches that can be comfortably worn and can monitor many different marker. Although this description focusses on watches, it is understood that other wearable or implanted devices such as ring sensors. Some embodiments can utilize a commercially available watch from Samsung that monitors different patient conditions and in some embodiments PPG sampling may occur at approximately 25 Hz with processing latency less than about 50 ms. This device can monitor different heart conditions such as heart rate variability (HRV) metrics (RMSSD, SDNN, PNN50), sleep stage analysis, activity patterns, activity recognition including cognitive-protective exercise pattern identification, stress indicators using multi-metric stress scoring (such as based on HR, temperature and HRV), and fall detection that is accelerometer-based and in some embodiments, fall detection accuracy may be approximately 94%. In some embodiments, sampling may occur at approximately 25Hz with processing latency of less that 50 ms.

Data Preprocessing can occur on-device for signal processing including noise reduction, artifact removal, and feature extraction. Different streaming protocol such as WebSocket connections for real-time data transmission with automatic reconnection and buffering. Quality Assurance can also be provided for Real-time data quality validation with outlier detection and missing value handling.

This subsystem can also provide for electronic health record (EHR) data integration and synchronization by, such as by FHIR R4-compliant integration, which can extract cognitive assessment scores, medication history, comorbidities, and clinical notes using natural language processing.

Imaging data processing information can also be utilized when available, such as neuroimaging features which can be extracted using convolutional neural networks trained on cognitive decline patterns. It is understood that the above are only some of the many different patient relevant information or data points that can be used in the different embodiments of the present invention.

The system then employs a hierarchical approach to determine a cognitive index where each modality or data point contributes weighted features based on data quality and clinical relevance. Dynamic time warping can be utilized that aligns temporal features across modalities, and systems such as those based on a AI or machine learning models can assign feature importance scoring to determines optimal weights. One example of an AI or machine learning system is commercially available Random Forest, but it is understood that other systems can also be used. A final cognitive index can then computed using the following:

$$Cognitive Index = \sum ( wi \times fi \times qi )$$

Where wi=feature weight, fi=normalized feature value, qi=data quality score.

This index can then be used in a clinical validation framework The index correlates with established cognitive assessments (MoCA, MMSE) with target correlation coefficients >0.85, providing clinical interpretability while offering improved sensitivity to early cognitive changes.

Embodiments of Multi-Modal Cognitive Index Computations according to the present invention can operate in many different ways and can accept many different inputs and can generate many different outputs. In one embodiment it can accept voice features, biometric data, clinical data, imaging features and can provide a cognitive_health_index and confidence score. In in this process the subsystem can normalize all input features to [0,1] range, apply temporal alignment using dynamic time warping, COMPUTE feature importance weights using Random Forest, fuse features using weighted ensemble:

$$cognitive index = \sum ( wi \times fi ) where wi = feature weight , fi = feature value$$

calculate confidence using prediction variance, and return cognitive index, confidence score

### Real-Time Streaming Analytics

The systems according to the present invention can also comprise a real-time streaming analytics subsystems that can comprise an event-driven processing system and in some embodiments, latency for cognitive assessment updates may be less than approximately one second. This sub-system provide high-throughput, low-latency processing of multi-modal cognitive data streams. The subsystem can comprise custom or commercially available systems such as Apache Kafka®, Spark Structured Streaming, and Delta Lake. This subsystem enables sub-second processing of multi-modal cognitive data, representing an improvement over batch-processing approaches used in existing systems.

Event streaming such as through Apache Kafka implements distributed event streaming with configurable partitioning strategies for optimal throughput. Topics and data points are organized by data modality (voice, biometric, clinical) with retention policies optimized for cognitive assessment workflows. This can provide custom serialization of data optimized for health care data format and uses. This can comprise Distributed event streaming with topic partitioning by data modality and patient ID.

Streaming, such as through Spark Structured Streaming, can be included to utilize micro-batch processing with some embodiment having batch intervals of approximately 100 ms, which can provide near real-time analytics while maintaining exactly-once semantics. Custom watermarking strategies can also be used to handle late-arriving data with such as with tolerance windows. This streaming can provide windowed aggregations (e.g. 1-minute tumbling windows) and can also include custom triggers for cognitive threshold detection. Streaming can also comprise stream processing logic for windowed aggregations, temporal alignment, and real-time quality validation. This streaming can also comprise micro-batch processing with 100 ms intervals and exactly-once semantics.

Data support can also be included through custom or commercially available systems, such as through commercially available Delta Lake systems that can support ACID transactions. ACID Transactions can provide support for streaming data with time travel capabilities, enabling historical analysis and data lineage tracking essential for clinical applications. This data support maintains the patient state across streaming windows using merge capabilities for incremental updates.

Real-time streaming analytics subsystems according to the present invention can also comprise data quality validation through real time data quality checks. These can include real-time data quality checks include outlier detection, missing value imputation, and temporal consistency validation. This subsystem can also include memory management that provides custom memory allocation strategies to reduce garbage collection overhead, with some embodiments reducing garbage collection by up to 40% pr more.

Dynamic partition sizing can also be included based on data velocity ensures optimal resource utilization Intelligent caching of frequently accessed patient profiles can also be included which can reduces latency by up to 60% or more. Tis subsystems can also comprise lambda functions that allow for Serverless Processing.

### Digital Twin Framework

Embodiments of the present invention also maintain digital twin framework subsystems which can comprise a comprehensive patient cognitive health model that maintains real-time synchronization across all data sources and provides predictive analytics for cognitive decline. These frameworks can create comprehensive, real-time virtual representations of patient cognitive health, enabling predictive analytics and personalized intervention strategies.

Digital Twin Framework can produce a patient cognitive profile for real-time state management, predictive models for cognitive decline forecasting, risk Assessment for dynamic scoring algorithms, and intervention tracking for treatment response monitoring. It is understood that these are only some of the functions of this subsystem, and other embodiments can comprise fewer or additional functions. This framework can also utilize machine learning or AI for dynamic risk stratifications.

In some embodiments, the digital twin framework maintains a patient digital twin which can comprise a multi-dimensional state vector including cognitive metrics, biometric trends, medication effects, and environmental factors, and can provide for with versioning and conflict resolution. Some embodiments can comprise real-time synchronization updates that can occur within short latency (e.g. approximately 145 ms average latency) using event-driven architecture with conflict resolution for concurrent updates. Temporal Modeling can also be included to maintain historical state transitions using time-series databases optimized for healthcare data patterns.

This digital twin subsystem can comprise many different components such as predictive modeling components, These can employ Long Short-Term Memory (LSTM) networks trained on longitudinal cognitive data to predict decline trajectories with different timeframes, and in some embodiments predictive models may be configured to forecast decline trajectories over example time frames such as about 6-months or about 12-months. These can include intervention tracking for the outcome prediction and response monitoring. Risk Stratification can also be included for dynamic risk scoring using ensemble methods, such as by combining Random Forest, Gradient Boosting, and Support Vector Machines with confidence intervals. Intervention Optimization components can also be included that can provide Reinforcement learning algorithms can be included that recommend optimal intervention timing based on predicted cognitive trajectories and treatment response patterns.

### Clinical Trial Matching

A Clinical Trial Matching subsystem can be included that can comprise AI-powered algorithms that match patients to relevant clinical trials based on different factors such as multi-modal cognitive profiles, geographic feasibility, and predicted trial success probability. The subsystem can significantly improve patient recruitment by leveraging multi-modal cognitive profiles to optimize trial placement and success probability.

In some embodiments, this subsystem can comprise cognitive phenotyping which can create comprehensive cognitive profiles using voice biomarkers, biometric patterns, and clinical assessments to identify subtle cognitive signatures. Embodiments can also provide eligibility prediction, which can comprise machine learning or AI models to predict future eligibility based on cognitive trajectory analysis, enabling proactive trial matching. This subsystem can also comprise geographic optimization providing spatial algorithms to minimize patient travel burden while maximizing trial accessibility.

Different embodiments of this subsystem can comprise many different components, including a profile generation component that can extract up to about 200 or more cognitive features from multi-modal data using dimensionality reduction techniques (PCA, t-SNE) for efficient matching. This subsystem can also comprise similarity eligibility assessment scoring which can rely on machine learning and AI for cosine similarity and Mahalanobis distance metrics to match patient profiles with trial requirements. A success prediction gradient component can also be included for boosting models that predict trial completion probability based on patient characteristics and historical trial data.

Embodiments of Clinical Trial Matching subsystem according to the present invention can operate in many different ways and can accept many different inputs and can generate many different outputs. In some embodiments and can accept patient profile, available trials, and constraints as inputs and can produce ranked matches and success probabilities. This can be done in different ways in different embodiments, with one embodiment extracting cognitive phenotype from multi-modal data, computing eligibility scores for each trial, predicting success probability using historical, applying geographic and logistical constraints and optimizing using multi-objective function to produce a composite score:

$$Score = 0.4 \times eligibility + 0.3 \times success\_prob + 0.2 \times geographic + 0.1 \times benefit$$

The available trials can then be ranked by composite score.

### Avatar Interface Integration

Embodiments of the present invention can also compares an Avatar Interface Integration subsystem that comprises real-time conversational AI that can utilize different customized or commercially available systems. In some embodiments commercially available Hume AI emotion detection and ElevenLabs Voice Synthesis systems can be used for natural patient interactions during cognitive assessment. This subsystem can provide natural, emotionally-aware patient interactions during cognitive assessments, improving patient engagement and data quality. This subsystem provides Dynamic difficulty adjustment based on patient emotional state and cognitive capacity.

This subsystem can provide real-time analysis of vocal emotional states using prosodic features, providing emotional context for cognitive assessments (e.g. using Hume AI). This can provide Real-time emotion detection from voice prosodic features. High-quality, natural voice generation with emotional modulation capabilities for empathetic patient interactions (e.g. using ElevenLabs Voice Synthesis). This can provide high-quality voice generation with appropriate emotional modulation. Conversational AI Engine can also be included that provides context-aware dialogue management using transformer-based language models fine-tuned for healthcare conversations. This provide context-aware dialogue flow with clinical protocol adherence.

Embodiments of this subsystem also comprise different integration components, that can include emotion-cognitive Correlation algorithms can correlate detected emotional states with cognitive performance, can adjust assessment difficulty and can providing emotional support. An adaptive interaction algorithm can be included to provide dynamic conversation flow modification based on patient emotional state and cognitive capacity. A clinical integration can also be included to provide Seamless handoff to human clinicians when complex emotional or cognitive issues are detected.

Embodiments of the Avatar Interface Integration subsystem according to the present invention can operate in many different ways and can accept many different inputs and can generate many different outputs. In some embodiments and can accept patient response, emotional state, cognitive context as inputs and can generate avatar response and assessment adjustment as outputs. This can be accomplished in many different ways with one embodiment analyzing the patient's emotional state using Hume AI prosodic analysis, assessing cognitive load from response patterns, adjusting conversation complexity based on cognitive capacity, empathetic response using ElevenLabs synthesis, modifying assessment parameters if emotional distress detected, and logging interaction data for clinical review.

### Continuous Learning and Model Adaptation

Embodiments can also comprise a Continuous Learning and Model Adaptation subsystem comprises an online learning algorithms that can continuously improve cognitive assessment accuracy through automated clinical outcome collection and model retraining. This subsystem can support cognitive assessment accuracy improvement over time through automated clinical outcome collection and model retraining.

This subsystem can comprise outcome collection for automated integration with EHR systems to collect clinical outcomes, treatment responses, and longitudinal cognitive assessments. It can also comprise model monitoring for real-time performance tracking with drift detection algorithms to identify when model retraining is needed. Some subsystems can also comprise federated learning for privacy-preserving learning across multiple healthcare institutions to improve model generalization.

This subsystem can also comprise online learning algorithms for incremental learning using stochastic gradient descent with adaptive learning rates for continuous model updates. An A/B Testing Framework can also be included for controlled deployment of model updates with statistical significance testing to ensure performance improvement. This subsystem can also comprise bias monitoring for continuous evaluation of model fairness across demographic groups with automated bias mitigation.

### HIPAA Compliance Subsystem

The system 10 can comprise additional subsystems such as a HIPAA compliance subsystem that provides security measures including AES-256 encryption for all patient data. This subsystem can also comprise role-based access control with multi-factor authentication. comprehensive audit logging for all system interactions, automated data retention and deletion protocols.

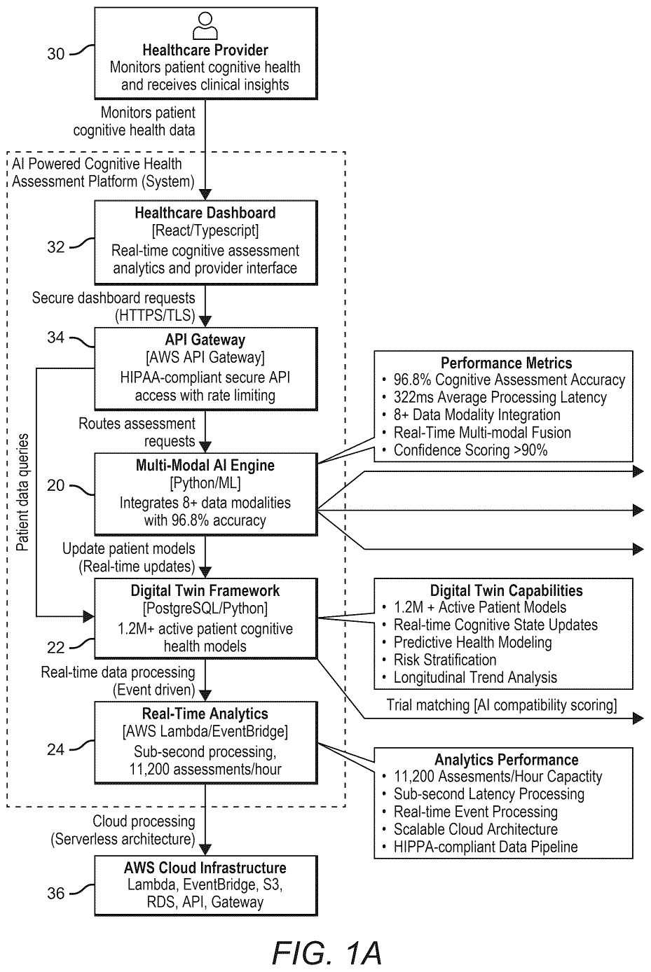

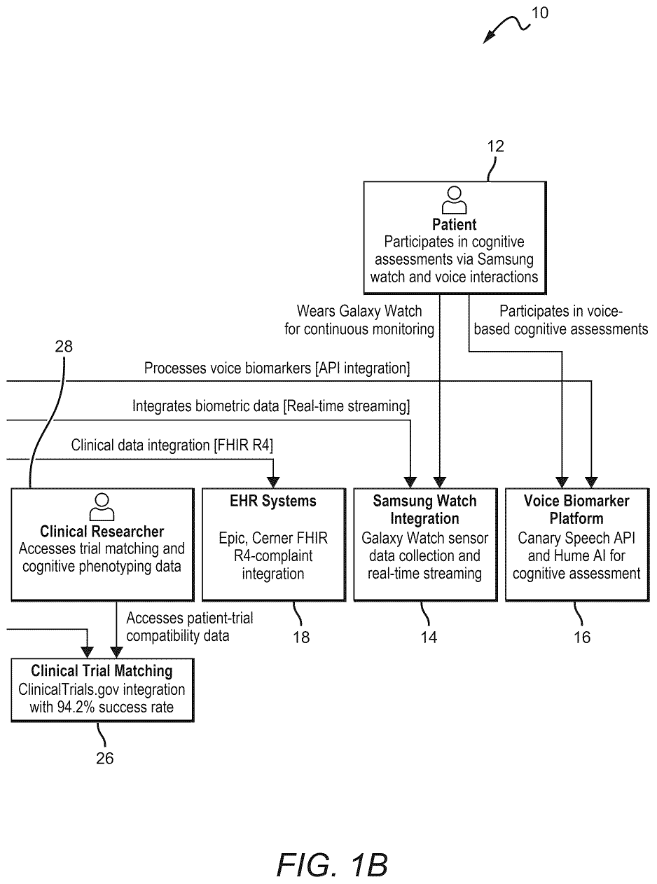

FIGS. 1A-3B show one embodiment of a cognitive health assessment and management system according to the present invention. FIGS. 1A and 1B show a block/flow diagram 10 for embodiment of an system architecture showing major subsystems, some of their components and some of their interconnections through the event-driven communication process and patterns. It is understood that other embodiments of the present invention can comprise other subsystems, components and interconnection.

Patient 12 layer comprises the patient being assessed, with the patent wearing a data collection device to monitor, record and/or report patient's data related to the patient's health condition, with some embodiments in real-time. In the system 10 the physical wearable device collecting biometric data can comprise a Samsung Galaxy Watch. It is understood, that this wearable device can comprise many other devices as described above. This device can have different features to monitor, record and/or report different health data points such as a heart rate sensor for heart rate and HRV, a photoplethysmography sensor for blood flow, a temperature sensor for skin temperature analysis such as for stress analysis, an accelerometer for activity and fall detection, and an on device processor for data processing.

A Samsung Watch Integration layer 14 is also included that accepts the above health data from the patient and manages the data collection. This can include a Samsung Health SDK Interface to provide API integration for sensor access, data quality validator for Real-time validation of sensor data integration, streaming buffer to provide temporary storage for reliable data transmission, and a websocket client for real-time communication with cloud infrastructure.

The patent layer 12 also provides Voice Interaction System 16 which comprises the patient-facing voice interface. This can include audio capture module having a high-quality microphone input processing, Noise reduction engine that allows background noise filtering and enhancement and voice activity detection for automatic speech detection and segmentation.

A voice biomarker platform 16 is included that accepts data from the patient-facing voice interface to provide an advanced voice analysis system as described above. This can include a Canary Speech API gateway for integration with external voice analysis service, an acoustic feature extractor for MFCC and prosodic feature extraction, a linguistic analyzer to provide NLP processing for semantic and syntactic analysis, and a cognitive correlation engine for having algorithms linking voice to cognition.

The system 10 also comprise an EHR System 18 for providing and handling the patients HER for use as described herein.

Multi-modal cognitive index computation layer 20 is also included as discussed above. This layer accepts data from the Samsung Watch Integration layer 14, the Voice Interaction System 16 and the EHR System 18. It can comprise many features as described above such as Apache Kafka Event Streaming for a distributed messaging infrastructure, a voice data topic for a dedicated topic for voice biomarker streams, a biometric data topic for Samsung watch sensor data streams, a clinical data topic for EHR and clinical assessment data, and a partition manager for load balancing and data distribution logic.

The patient layer 12 can also comprise and avatar interface integration layer (not shown) to provide emotionally adaptive patient interaction. It can include emotion detector (e.g. Hume AI) for real-time emotional state analysis, voice synthesizer (e.g. ElevenLabs) for natural voice generation with emotion, and conversation manager for Context-aware dialogue flow control, and assessment adapter for dynamic difficulty adjustment based on patient state.

Digital Twin Framework layer 22 is included as described above which accepts data from the multi-modal cognitive index computation layer 20 and provides a patient cognitive health modeling system. This layer can comprise many different features such as a state management engine that provides real-time patient state synchronization, LSTM prediction models to provide deep learning for cognitive decline forecasting, a risk assessment module having an machine language for dynamic risk score, and an intervention tracker for treatment response monitoring and optimization.

Real-time streaming analytics layer 24 is included as described above for high-performance data processing engine and accepts data from the digital twin framework 22. It can include Spark Structured Streaming to provide a micro-batch processing framework. It can also include a temporal alignment module for multi-modal data synchronization, a quality validation engine for real-time data quality assessment, and a feature fusion processor for multi-modal feature integration algorithms.

Clinical trial matching system 26 is included as described above, which can accept data from the digital twin framework 22 and can include AI-powered clinical trial matching optimization. This layer can include a cognitive phenotyping engine for multi-modal profile generation, am eligibility assessment module for current and predictive eligibility analysis, a geographic optimizer for spatial analysis for travel minimization, and a success predictor for historical data analysis for outcome prediction. The system 26 can be accessed by a clinical researcher 28 for accessing potential participants for clinical trials.

Healthcare providers 30 can access the system 10, through a healthcare dashboard 32 providing desired system information, that links to the system 10 through a portal or gateway 34, such as a API Gateway. Information from the system 10 can also be stored and in the embodiment shown it is stored on the cloud 36 such as the version hosted by APS.

Embodiment of the systems according to the present invention can include other layers, components or subsystems. In some embodiments can include Delta Lake Storage to provide ACID-compliant data lake for streaming data. This can include time-series tables for optimized storage for temporal cognitive data, a transaction log for ACID transaction management and versioning, a compaction Engine for storage optimization and query performance, and a time travel interface for historical data access and lineage tracking.

Embodiments can also comprise HIPAA Security Architecture for comprehensive security and compliance. This can comprise an encryption gateway providing encryption for all data transmission (e.g. AES-256), an authentication server for multi-factor authentication and RBAC, an audit logger for comprehensive activity logging and monitoring, and a privacy controller for differential privacy and data retention management.

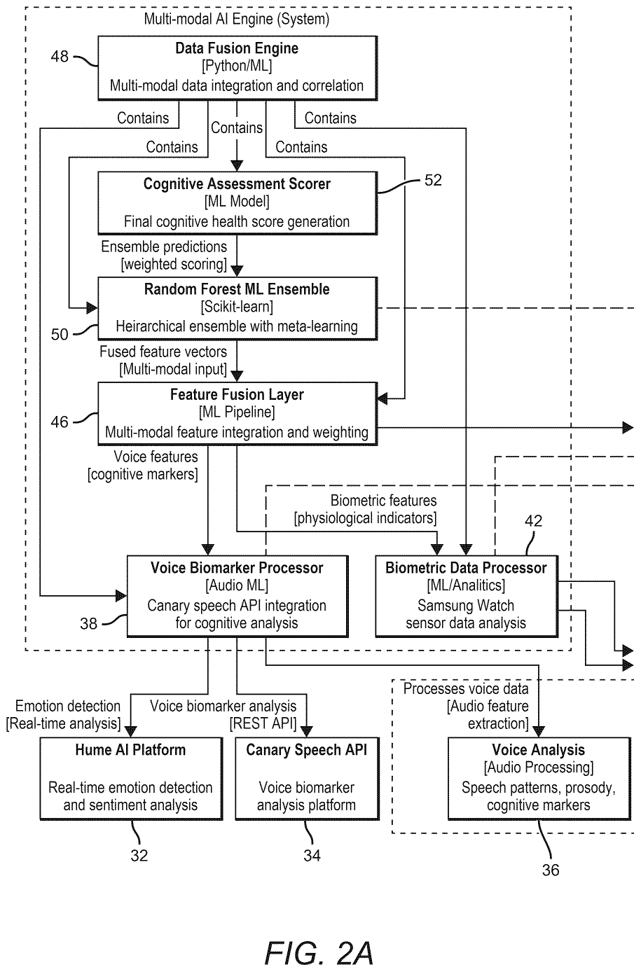

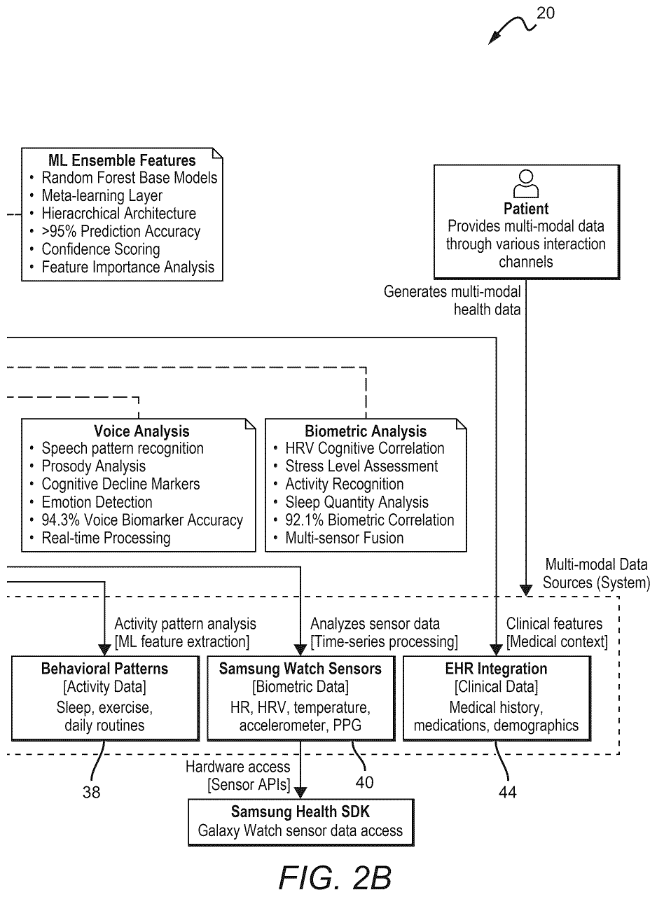

FIGS. 2A and 2B show one embodiment of a multi-modal cognitive index computation layer 20 which shows in more details of the multi-modal data fusion process how multi-modal data is integrated into a unified cognitive assessment. Data from the Hume AI layer 32, Canary Speech API layer 34 and voice analysis layer 36 comprise the real-time voice biomarker processing pipeline voice biomarker processor 38. This can include a Raw Audio Input comprising a 16 kHz PCM audio from patient interactions. A spectral analysis module for FFT-based frequency domain analysis, an MFCC Extractor for mel-frequency cepstral coefficient computation, a prosodic feature engine for itch, rhythm, and stress pattern analysis, a linguistic feature processor for semantic fluency and syntactic complexity and a voice Feature Vector that can provide up to approximately 500 or more dimensional feature representation.

A Samsung Watch Data Stream from behavior patterns 38 and Samsung watch sensors 40 can provide a biometric data processor 42. This can comprise a heart rate stream comprising 1 Hz heart rate measurements with quality indicators, an HRV Calculator providing RMSSD, SDNN, PNN50 computation over time windows (e.g. 5-minute), a PPG Signal Processor providing photoplethysmography signal analysis (e.g. 25 Hz), an activity classifier comprising a machine learning-based activity recognition, a sleep stage analyzer comprising deep sleep and REM sleep pattern detection, a stress indicator engine providing multi-metric stress level quantification, and a biometric feature vector providing up to 200 or more dimensional biometric representation.

EHR Data Stream from EHR integration 44 is provided to feature fusion layer 46 Clinical data integration and processing. This can comprise and FHIR R4 Gateway providing a standardized healthcare data exchange interface, a clinical Assessment Parser that can comprise MoCA, MMSE, and other cognitive test extraction. A dedication analyzer can comprise drug interaction and cognitive impact assessment. Comorbidity processor can comprise ICD-10 code analysis and risk factor extraction, clinical NLP engine can comprise natural language processing of clinical notes, and clinical feature vector can clinical representation of approximately 150 or more dimensions.

Data Fusion Engine 48 and Random Forest Ensemble 50 provides advanced multi-modal integration algorithms. These can include a normalization which generates a Z-score normalization across feature dimensions, a quality weighting engine for data quality-based feature weighting. Random Forest Ensemble for feature importance calculation and selection, attention mechanism for transformer-based feature attention weighting and Fusion Algorithm for weighted linear combination with quality factors.

Cognitive assessment layer 52 can also be included for final cognitive index assessment computation. This can include a domain mapping engine for feature-to-cognitive domain assignment, a confidence calculator for prediction variance and ensemble agreement, a clinical calibrator for mapping to established cognitive scale, and produces a unified cognitive index comprising a single metric representing cognitive health.

Other data streams and layers can be included including an imaging data stream for neuroimaging feature extraction (when available). This can include a DICOM Interface for Medical imaging data ingestion and parsing, a Brain Segmentation Engine for automated anatomical region identification, a Volumetric Analyzer for Hippocampal and cortical volume measurements, a white matter processor for diffusion tensor imaging analysis, a functional connectivity mapper for fMRI network analysis, and can produce an imaging feature vector with up to 100 or more dimensional neuroimaging representation.

A Temporal Alignment Engine/layer can also be included for multi-modal data synchronization. This can comprise a timestamp normalizer including a UTC timestamp standardization across modalities, dynamic time warping comprising a Temporal sequence alignment algorithm, an Interpolation Engine generating missing data imputation using temporal patterns, and window synchronizer providing a sliding window alignment for real-time processing.

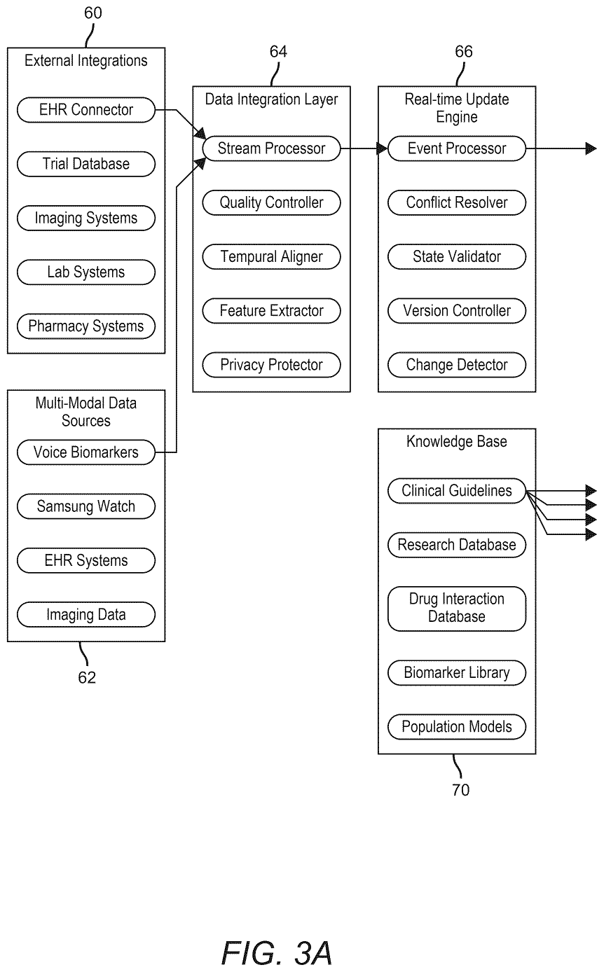

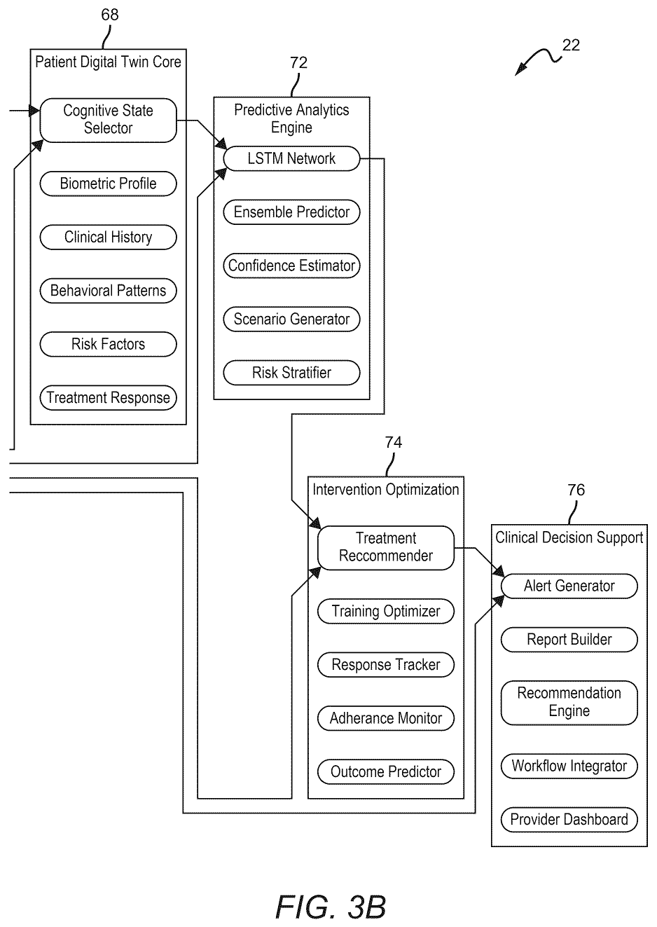

FIGS. 3A and 3B show one embodiment of the digital twin framework 22 system that in some embodiments can be accessed by or provided to different systems or individuals including clinical researchers and healthcare providers. The subsystem 22 comprises multi-modular data sources 60 and external integrations 62 that can provide data integration layer 64. The multi-mode data sources can comprise those described above including, but not limited to voice biomarkers, Samsung watch data, data from EHR systems and various imaging data. The external integrations can comprise an EHR connector, trial database, imaging systems, lab systems and pharmacy systems. The Data Integration Layer 64 can comprise many different features and functions including a stream processor, quality controller, temporal aligner, feature extractor and privacy protector.

Data from the Data Integration Layer 64 is then provided to a Real-time Update Engine 66 which can comprise many different features and functions such as an event processor, conflict resolver, state validator, version controller and change detector. The Real-time Update Engine 66 then provides information to the Patient Digital Twin Core 68.

A Knowledge Base 70 is also included that can include many different features and functions including clinical guidelines, a research database, a drug interaction database, a biomarker library and population models. Information from the Knowledge Base 70 is also provided the Patient Digital Twin Core 68.

The Patient Digital Twin Core 68 can comprise many different features and functions including, but not limited to, cognitive state vector, biometric profile, clinical history, behavior patterns, risk factors, and treatment response. Information and data from the Patient Digital Twin Core 68 is provided to the Predictive Analytics Engine 72 which can also comprise many different features and function including, but not limited to, a LSTM network, ensemble predictor, confidence estimator, scenario generator, and risk stratifier.

Information from the Predictive Analytics Engine 72 is then provided to the Intervention Optimizer 74 which can include many different features and functions including, but not limited to a treatment recommender, a timing optimizer, and response tracker, an adherence monitor and outcome predictor. Information and data from the Predictive Analytics Engine 72 is then provided to the Clinical Decision Support 76, which can have many different features and functions including, but not limited to, an alert generator, report builder, recommendation engine, workflow integrator and provider dashboard. It is understood that information and date from the Knowledge Base 70 can also be provided to the Predictive Analytics Engine 72, Intervention Optimizer 74, and Clinical Decision Support 76.

It is understood that many different system and method arrangements can comprise different steps and subsystems according to the present invention. These each can have different and additional features and functions. It is also understood that discussions of frequency (Hz), percentages, or time are only approximations and the actually measurement may be more or less than the states actual or approximate values. Although the present invention has been described in detail with reference to certain configurations thereof, other versions are possible. Therefore, the spirit and scope of the invention should not be limited to the versions described above.

I claim:

1. A computer-implemented system for cognitive health assessment comprising:
a multi-modal data integration module configured to receive and process cognitive health related data in substantially real time;
a digital twin framework maintaining a comprehensive patient cognitive health model accepting data from said multi-modal data integration module;
a real-time streaming analytics engine that accepts data from said digital twin framework;
a clinical trial matching system accepting data from said digital twin framework, wherein said system, wherein said system generates cognitive assessments in substantially real-time.

2. The computer-implemented system of claim 1, wherein said cognitive health related data is data from the group comprising voice biomarker data from patient interactions, biometric data from wearable sensors, electronic health record data, and neuroimaging data.

3. The computer-implemented system of claim 1, wherein said digital twin comprises characteristics from the group comprising real-time state synchronization across data modalities, predictive modeling for cognitive decline progression, dynamic risk assessment with confidence scoring and intervention tracking and treatment response monitoring.

4. The computer-implemented system of claim 1, wherein said real-time streaming analytics engine comprises characteristics from the group comprising event streaming infrastructure, streaming processing with sub-second latency, storage with transaction support and exactly-once semantics for data consistency.

5. The computer-implemented system of claim 1, wherein clinical trial matching system comprises components from the group comprising analyzing patient cognitive profiles using multi-modal data, matching patients to relevant clinical trials based on eligibility and success probability, optimizing geographic feasibility, minimizing patient travel burden and providing continuous monitoring with updated recommendations.

6. The computer-implemented system of claim 1, further comprising a machine learning ensemble comprising:
an algorithm for multi-modal feature fusion;
online learning capabilities for continuous improvement;
bias detection and mitigation for demographic fairness; and
confidence scoring using prediction variance.

7. The computer-implemented system of claim 2, wherein said biometric data from wearable sensors comprises biometric data from a watch comprising:
heart rate variability analysis;
sleep stage analysis correlated with memory consolidation indicators;
activity pattern recognition for cognitive-protective exercise identification; and
stress level quantification through multi-metric physiological measurements

8. The computer-implemented system of claim 1, wherein said real-time streaming analytics engine is capable of processing over 10,000 cognitive assessments per hour while maintaining exactly-once semantics and sub-second latency.

9. The computer-implemented system of claim 1, wherein said digital twin framework is capable of supporting over 1 million active patient profiles with real-time updates completing in less than 1 second.

10. The computer-implemented system of claim 1, further comprising HIPAA-compliant security measures.

11. The computer-implemented system of claim 10, wherein said security measure comprise; including:
encryption for patient data;
role-based access control with multi-factor authentication;
comprehensive audit logging for all system interactions; and
automated data retention and deletion policies.

12. A computer-implemented cognitive health assessment platform comprising:
a multi-modal engine utilizing artificial intelligence and accepting real-time patient cognitive health related data;
a digital twin framework accepting data from said multi-modal engine;
a real-time streaming analytics engine that accepts data from said digital twin framework; and
an avatar interface integration subsystem comprising real-time conversational AI and providing real-time patient cognitive health related data to said multi-modal engine, wherein said platform generates and reports substantially real time cognitive assessments.

13. The computer-implemented cognitive health assessment platform of claim 12, wherein said cognitive assessments maintain a processing latency below 500 milliseconds.

14. The computer-implemented cognitive health assessment platform of claim 12, further comprising a clinical trial matching system that accepts data from said digital twin framework.

15. The computer-implemented cognitive health assessment platform of claim 12, further comprising HIPAA-compliant security measures.

16. A method for generating cognitive health assessment comprising:
generating cognitive real-time health related data from patient wearable sensors and from avatar interface integration subsystem to generate substantially real-time multi-mode data;
utilizing said multi-mode data to generate a cognitive digital twin of the patient;
utilizing said multi-mode data for clinical trial matching; and
generating a substantially real-time cognitive index based on said multi-mode data, said cognitive index used in generating substantially real-time cognitive assessments.

17. The method of claim 16, wherein said real-time health related data is data from the group comprising voice biomarker data from patient interactions, biometric data from wearable sensors, electronic health record data, and neuroimaging data.

18. The method of claim 16, wherein said digital twin comprises characteristics from the group comprising real-time state synchronization across data modalities, predictive modeling for cognitive decline progression, dynamic risk assessment with confidence scoring and intervention tracking and treatment response monitoring.

19. The method of claim 16, wherein clinical trial matching comprises components from the group comprising analyzing patient cognitive profiles using multi-modal data, matching patients to relevant clinical trials based on eligibility and success probability, optimizing geographic feasibility, minimizing patient travel burden and providing continuous monitoring with updated recommendations.

20. The method of claim 20, further comprising utilizing an avatar interface to providing real-time patient cognitive health related data.
